(19) United States
(12) Patent Application Publication
(10) Pub. No.: US 2026/0241139 A1
(43) Pub. Date: Aug. 20, 2026

(54) CATHETER ASSEMBLY

(71) Applicant: ConvaTec Limited, Flintshire (GB)

(72) Inventors: Marian Novak, Trebisov (SK); Zac Urquhart, Wirral (GB); Kate Duhig, Chester (GB)

(21) Appl. No.: 19/644,292

(22) Filed: Apr. 10, 2026

Related U.S. Application Data
(63) Continuation of application No. PCT/GB2024/052511, filed on Sep. 30, 2024.
(60) Provisional application No. 63/589,098, filed on Oct. 10, 2023.

(30) Foreign Application Priority Data
Dec. 12, 2023 (GB) 2318908.7

(51) Int. Cl. A61M 25/00

(52) U.S. Cl. CPC A61M 25/002; A61M 2207/00; A61M 2209/06

(57) ABSTRACT

A catheter assembly comprising a case comprising outer (20), inner (40) and intermediate (60) tubular portions, wherein the intermediate tubular portion (60) is comprised of a softer material than the outer (20) and inner (40) tubular portions.

### TECHNICAL FIELD OF THE INVENTION

The present invention relates to catheter assemblies, in particular, although not exclusively to urinary catheters and especially intermittent female urinary catheters.

### BACKGROUND TO THE INVENTION

A catheter is a medical device comprising a hollow catheter tube designed for insertion into canals, vessels, passageways or body cavities to permit injection, drainage or withdrawal of fluids or substances therefrom, or to ensure said canals, vessels, passageways etc. remain open. Urinary catheters are designed for insertion into a user's bladder via the urethra to drain the bladder.

To maximise comfort and minimise the risk of trauma and/or infection, an outer surface of the catheter tube is typically wetted using a wetting agent prior to insertion by the user. The wetting agent may be a lubricant, such as a lubricant gel. In further developments, the catheter tube itself comprises, is integrated with or is coated with a hydrophilic component (e.g. a hydrophilic polymer) which is activated to reduce friction further upon application of the wetting agent, which may for example be water.

Some catheters may be supplied pre-wetted (or lubricated) in a packaging, for instance, where the catheter is at least partially submerged within wetting agent or lubricant within the packaging. Whilst this may ensure the catheter tube is adequately wetted or lubricated prior to use, such arrangements suffer in that components of the catheter other than the catheter tube such as a gripper element or funnel can also become wetted or coated in lubricant. This has a detrimental effect of the experience of the user where it may become difficult to hold and direct the catheter tube as required. This is particularly problematic where the user is performing self-catheterisation. Further, having the catheter submerged may effectively reduce the shelf-life of the catheter due to long-term exposure of components of the catheter to moisture.

It is therefore seen advantageous to provide a catheter which may be wetted or lubricated at or immediately prior to the point of use.

In an attempt to address this, some catheters are provided in packaging which includes a rupturable container or sachet within the packaging which a user may burst to release the wetting agent. Typically, this involves the user squeezing the packaging to cause the container/sachet to break. However, such arrangements can experience similar problems to those discussed above where the wetting agent is allowed to come into contact with other components of the catheter. Such arrangements also result in the possibility of the catheter tube not being fully wetted, or indeed wetted at all, prior to use. This can be harmful for the user.

It is therefore advantageous to provide a catheter which includes a means of supplying a wetting agent solely to the catheter tube to improve user experience.

In further prior art solutions, the catheter may be packaged within a packaging which includes a wetting device. In use, the catheter tube may be moved through the wetting device as the catheter is removed from the packaging and in doing so wetting the catheter tube. Examples of such catheters are shown in PCT application No. PCT/IB2018/001539 in the name of ConvaTec Limited.

Furthermore, intermittent catheters may be carried by the user for use when required, it is desirable that the catheter be discrete, such that it may not readily be identified as a piece of medical equipment. To address this, some catheters are provided in a case where they are stored prior to use, this conceals the catheter but may also protect it. However, cases can be complex to assemble and or to access the catheter for use.

It is an aim of an embodiment or embodiments of the invention to overcome or at least partially mitigate one or more problems with the prior art and/or to provide an improved catheter assembly.

### SUMMARY OF THE INVENTION

The present invention provides a catheter assembly according to the appended claims.

An aspect of the present disclosure provides a catheter assembly. The catheter assembly may comprise an inner tubular portion. The catheter assembly may comprise an intermediate tubular portion. The catheter assembly may comprise an outer tubular portion. The inner and intermediate tubular portions may be radially nested. The inner and outer tubular portions may be radially nested. The intermediate and outer tubular portions may be radially nested. The inner, intermediate and outer tubular portions may be radially nested. The intermediate tubular portion may be arranged between the inner and outer tubular portions. The intermediate tubular portion may be adjacent to the inner tubular portion. The intermediate tubular portion may be adjacent to the outer tubular portion. The catheter assembly may comprise a case. The case may comprise one or more or each of the inner, intermediate and/or outer tubular portions. The inner tubular portion may comprise a first material. The intermediate tubular portion may comprise a second material. The outer tubular portion may comprise a third material. The second material may be softer than the first material. The second material may be softer than the third material.

Accordingly, a first aspect of the present disclosure provides a catheter assembly comprising radially nested inner, intermediate and outer tubular portions, wherein the intermediate tubular portion is arranged between and adjacent to the inner and outer tubular portions, the inner tubular portion comprising a first material, the intermediate tubular portion comprising a second material and the outer tubular portion comprising a third material, wherein the second material is softer than the first and third materials.

Preferably a case comprises the inner tubular portion, outer tubular portion and intermediate tubular portion. Accordingly, a further aspect of the present disclosure provides a catheter assembly comprising a case, the case comprising radially nested inner, intermediate and outer tubular portions, wherein the intermediate tubular portion is arranged between and adjacent to the inner and outer tubular portions, the inner tubular portion comprising a first material, the intermediate tubular portion comprising a second material and the outer tubular portion comprising a third material, wherein the second material is softer than the first and third materials.

In this context “softer” means having a lower Shore hardness as measured according to ISO 868.

Advantageously, by providing a softer intermediate tubular portion between the inner and outer tubular portions, the inner and outer tubular portions can more easily be joined together to form the catheter assembly, in particular the case thereof. In order to protect the catheter, the case will typically be made of a relatively hard material, inserting the inner portion into the outer portion will typically require some elastic deformation to engage a snap-fit or similar joining mechanism which holds the inner and outer tubular parts together. The harder the material, the greater the force required, and also the increased risk that a component may be damaged. By using a softer intermediary, the softer material can be deformed to engage with the inner and outer portions with a reduced force.

Accordingly, in a preferred embodiment there is provided a catheter assembly comprising radially nested inner, intermediate and outer tubular portions, wherein the intermediate tubular portion is arranged between and adjacent to the inner and outer tubular portions, the inner tubular portion comprising a first material, the intermediate tubular portion comprising a second material and the outer tubular portion comprising a third material, wherein the second material is softer than the first and third materials, wherein a case comprises the inner, intermediate and outer tubular portions and the case is configured to receive a catheter.

One of the outer tubular portion and the intermediate tubular portion may comprise one or more depressions. The one or more depressions may be grooves, recesses, bowls dips, dimples, or the like. The depressions may be provided on a radially inner wall of the outer tubular portion. The depressions may extend radially into the outer tubular portion. The depressions may extend circumferentially around the outer tubular portion. The other of the outer tubular portion and the intermediate tubular portion may comprise one or more projections. The one or more projections may be arranged in the depressions. The one or more projections may be ribs, rings, or may be pips, pimples, pins, fins, or the like. The projections may project radially from the intermediate tubular portion. The projections may be provided on a radially outer wall of the intermediate tubular portion. The projections may extend circumferentially around the intermediate tubular portion. The projections may project radially inward from the outer tubular portion. The projections may be provided on a radially inner wall of the outer tubular portion. The projections may extend circumferentially around the outer tubular portion. For example, the projections may extend radially outward from the intermediate tubular portion, into depressions extending radially into the outer tubular portion.

Advantageously, the provision of corresponding projections and recesses on the outer and intermediate tubular portions ensures the two components remain correctly positioned.

The inner tubular portion may comprise a support. The support may be aligned with the depression and projection. The support may be axially aligned with the depression and projection. The support may be provided on a radially outer wall of the inner tubular portion. The support may comprise one or more projections. The projections may be ribs, or may be pips, pins, fins, or the like. The support may extend axially along the inner tubular portion. The support may extend circumferentially around the inner tubular portion. The support may project radially outward from the radially outer wall of the inner tubular portion.

Advantageously, by providing on or more supports on the inner tubular portion the connection between the intermediate and outer tubular portions can be reinforced whilst also reducing the surface area, and thus friction of the connection between the inner and intermediate portions. That is, the contact between the inner and intermediate tubular portions is limited to radially outer faces of the supports, so the effective contact area between the inner and intermediate tubular portions is reduced, thereby reducing area upon which friction acts. Thus as the inner tubular portion is inserted the force required is reduced it also reduces the risk that the intermediate portion could “snag” and be displaced by the insertion of the inner tubular portion. It therefore allows the inner tubular portion to support the portions of the intermediate tubular portion to improve the connection between the intermediate and outer tubular portions.

Accordingly, in a preferred embodiment there is provided a catheter assembly comprising radially nested inner, intermediate and outer tubular portions, wherein the intermediate tubular portion is arranged between and adjacent to the inner and outer tubular portions, the inner tubular portion comprising a first material, the intermediate tubular portion comprising a second material and the outer tubular portion comprising a third material, wherein the second material is softer than the first and third materials, wherein one of the outer tubular portion and intermediate tubular portion comprises one or more depressions and the other of the outer tubular portion and intermediate tubular portion comprises one or more projections, the projections arranged in the depressions and wherein the inner tubular portion comprises at least one support aligned with the depression and projection.

The case may be suitable for housing an intermittent catheter. The catheter may be a female intermittent catheter. The catheter may have a length of between 90 mm to 200 mm. The catheter may have a length of between 100 mm and 150 mm or for example between 130 mm and 155 mm, such as about 135 mm. The catheter assembly may have a length corresponding to the length of the catheter. For example, the length of the catheter assembly, i.e. the closed length of the casing may be between 2 mm and 20 mm, for example between 2 mm and 15 mm, such as between 2 mm and 10 mm longer than the length of catheter The catheter assembly (when closed) may have a length of 10 and 25 cm; it may have a length of between 11 and 16 cm, for example, between 140 mm and 165 mm, such as 142 mm.

The outer tubular portion may be comprised of plastic. The outer tube may be comprised of a plastic with a Shore hardness as measured using a Shore Durometer “A” of between 70 and 95 (i.e. between 70A and 95A). The outer tube may be comprised of a plastic with a Shore hardness as measured using a Shore Durometer “A” of between 70 and 80 (i.e. between 70A and 80A). The outer tube may be comprised of a plastic with a Shore hardness as measured using a Shore Durometer “A” of between 80 and 85 (i.e. between 80A and 85A). The outer tube may be comprised of a plastic with a Shore hardness as measured using a Shore Durometer “A” of between 85 and 90 (i.e. between 85A and 90A). The outer tube may be comprised of a plastic with a Shore hardness as measured using a Shore Durometer “A” of between 90 and 95 (i.e. between 90A and 95A). The outer tube may be comprised of a plastic with a Shore hardness as measured using a Shore Durometer “A” of between 85 and 95 (i.e. between 85A and 95A). The outer tube may be comprised of a plastic with a Shore hardness as measured using a Shore Durometer “D” of between 40 and 90 (i.e. between 40D and 90D). The outer tube may be comprised of a plastic with a Shore hardness as measured using a Shore Durometer “D” of between 50 and 80 (i.e. between 50D and 80D). The outer tube may be comprised of a plastic with a Shore hardness as measured using a Shore Durometer “D” of between 60 and 70 (i.e. between 60D and 70D). The outer tube may be comprised of a plastic with a Shore hardness as measured using a Shore Durometer “D” of between 40 and 50 (i.e. between 40D and 50D). The outer tube may be comprised of a plastic with a Shore hardness as measured using a Shore Durometer “D” of between 50 and 60 (i.e. between 50D and 60D). The outer tube may be comprised of a plastic with a Shore hardness as measured using a Shore Durometer “D” of between 70 and 80 (i.e. between 70D and 80D). The outer tube may be comprised of a plastic with a Shore hardness as measured using a Shore Durometer “D” of between 80 and 90 (i.e. between 80D and 90D). The outer tube may be comprised of a plastic with a Shore hardness as measured using a Shore Durometer “A” of between 80 and 95 (i.e. between 80A and 95A), and/or as measured using a Shore Durometer “D” of between 40 and 90 (i.e. between 40D and 90D). The outer tubular portion may be comprised of polyvinylchloride, or polypropylene. The outer tubular portion may be comprised of the same material as the inner tubular portion.

In a preferred embodiment, there is provided a catheter assembly comprising radially nested inner, intermediate and outer tubular portions, wherein the intermediate tubular portion is arranged between and adjacent to the inner and outer tubular portions, the inner tubular portion comprising a first material, the intermediate tubular portion comprising a second material and the outer tubular portion comprising a third material, wherein the second material is softer than the first and third materials, wherein the inner and/or outer tubular portions have a Shore hardness of between hardness as measured using a Shore Durometer “A” of between 80 and 95 (i.e. between 80A and 95A), and/or as measured using a Shore Durometer “D” of between 40 and 90 (i.e. between 40D and 90D).

The external housing may be manufactured, imported and sold independently of the catheter. The catheter assembly may comprise a catheter. The catheter may comprise a catheter tube. The catheter tube may have an insertion end for inserting into a patient and an outlet end. The outlet end may comprise one or more external handling features. The catheter may comprise an insertion end for inserting into the patient and an outlet end from which fluid is excreted during use. The outlet end may comprise one or more flow enhancing features such as a funnel which diverges along the flow direction. The outlet end may comprise an external handling surface. The external handling surface may be exposed for a user to handle when a/the cap is removed. The external handling surface may comprise one or more surface features to enhance a user's grip. The one or more surface features may comprise one or more grooves. The catheter tube may comprise one or more inlets for receiving urine at an insertion end thereof. The insertion end of the catheter may define a proximate end of the catheter assembly. The outlet end of the catheter may define a distal end of the catheter assembly.

The catheter may comprise a main flow path for the passage of urine. The main flow path may extend along and define a longitudinal axis of the catheter. The main flow path may be provided by a wall of catheter tube. The main flow path may have a proximal inlet at an insertion end of the catheter, and a distal outlet.

The inner tubular portion may comprise a catheter holder. A radially inner wall of the inner tubular portion may be configured to receive a catheter. The inner tubular portion comprise an end wall. The end wall may extend radially inward from a proximal end of the inner tubular portion.

According to a preferred embodiment, there is provided a catheter assembly comprising radially nested inner, intermediate and outer tubular portions, wherein the intermediate tubular portion is arranged between and adjacent to the inner and outer tubular portions, the inner tubular portion comprising a first material, the intermediate tubular portion comprising a second material and the outer tubular portion comprising a third material, wherein the second material is softer than the first and third materials, wherein the inner tubular portion comprises a catheter holder.

The end wall may be provided with an aperture extending therethrough. The aperture may have a diameter larger than the diameter of a/the catheter tube. The aperture may be at least 0.1 mm greater in diameter than a/the catheter tube. The aperture may be at least 0.2 mm greater in diameter than a/the catheter tube. The aperture may be at least 0.3 mm greater in diameter than a/the catheter tube. The aperture may be at least 0.4 mm greater in diameter than a/the catheter tube. The aperture may be at least 0.5 mm greater in diameter than a/the catheter tube. The aperture may be no more than 0.1 mm greater in diameter than a/the catheter tube. The aperture may be no more than 0.2 mm greater in diameter than a/the catheter tube. The aperture may be no more than 0.3 mm greater in diameter than a/the catheter tube. The aperture may be no more than 0.4 mm greater in diameter than a/the catheter tube. The aperture may be no more than 0.5 mm greater in diameter than a/the catheter tube. The aperture may be no more than 1 mm greater in diameter than a/the catheter tube. The aperture may be between 0.01 and 0.2 mm greater in diameter than a/the catheter tube. The aperture may be between 0.03 and 0.18 mm greater in diameter than a/the catheter tube. The aperture may be between 0.04 and 0.16 mm greater in diameter than a/the catheter tube. The aperture may be between 0.05 and 0.15 mm greater in diameter than a/the catheter tube. Preferably the aperture may be between 0.06 and 0.1 mm greater in diameter than a/the catheter tube. More preferably the aperture may be between 0.06 and 0.08 mm greater in diameter than a/the catheter tube. For example, the aperture may be 0.07 mm greater in diameter than a/the catheter tube.

According to a preferred embodiment, there is provided a catheter assembly comprising radially nested inner, intermediate and outer tubular portions, wherein the intermediate tubular portion is arranged between and adjacent to the inner and outer tubular portions, the inner tubular portion comprising a first material, the intermediate tubular portion comprising a second material and the outer tubular portion comprising a third material, wherein the second material is softer than the first and third materials, wherein the inner tubular portion comprises an end wall, the end wall extending radially inward from a proximal end of the inner tubular portion, the end wall being provided with an aperture extending therethrough.

By providing an aperture larger than the catheter tube, a consistent layer of lubricant/wetting agent can be applied to catheter tube.

The aperture may be provided with one or more projections, for example 2, or more preferably 3 or more projections. The one or more projections may be ribs, or may be pips, pins, fins, or the like. The projections may be semi-spherical pips. The projections may be equally spaced about the aperture. The projections may be configured to bear against the a/the catheter tube.

The aforementioned projections further improve the distribution of wetting agent/lubricant on the catheter tube, ensuring the catheter is centrally located in the aperture.

The inner tubular portion may be comprised of plastic. The inner tube may be comprised of a plastic with a Shore hardness as measured using a Shore Durometer “A” of between 70 and 95 (i.e. between 70A and 95A). The inner tube may be comprised of a plastic with a Shore hardness as measured using a Shore Durometer “A” of between 70 and 80 (i.e. between 70A and 80A). The inner tube may be comprised of a plastic with a Shore hardness as measured using a Shore Durometer “A” of between 80 and 85 (i.e. between 80A and 85A). The inner tube may be comprised of a plastic with a Shore hardness as measured using a Shore Durometer “A” of between 85 and 90 (i.e. between 85A and 90A). The inner tube may be comprised of a plastic with a Shore hardness as measured using a Shore Durometer “A” of between 90 and 95 (i.e. between 90A and 95A). The inner tube may be comprised of a plastic with a Shore hardness as measured using a Shore Durometer “A” of between 85 and 95 (i.e. between 85A and 95A). The inner tube may be comprised of a plastic with a Shore hardness as measured using a Shore Durometer “D” of between 40 and 90 (i.e. between 40D and 90D). The inner tube may be comprised of a plastic with a Shore hardness as measured using a Shore Durometer “D” of between 50 and 80 (i.e. between 50D and 80D). The inner tube may be comprised of a plastic with a Shore hardness as measured using a Shore Durometer “D” of between 60 and 70 (i.e. between 60D and 70D). The inner tube may be comprised of a plastic with a Shore hardness as measured using a Shore Durometer “D” of between 40 and 50 (i.e. between 40D and 50D). The inner tube may be comprised of a plastic with a Shore hardness as measured using a Shore Durometer “D” of between 50 and 60 (i.e. between 50D and 60D). The inner tube may be comprised of a plastic with a Shore hardness as measured using a Shore Durometer “D” of between 70 and 80 (i.e. between 70D and 80D). The inner tube may be comprised of a plastic with a Shore hardness as measured using a Shore Durometer “D” of between 80 and 90 (i.e. between 80D and 90D). The inner tube may be comprised of a plastic with a Shore hardness as measured using a Shore Durometer “A” of between 80 and 95 (i.e. between 80A and 95A), and/or as measured using a Shore Durometer “D” of between 40 and 90 (i.e. between 40D and 90D). The inner tube may be comprised of polyvinylchloride, or polypropylene. The inner tubular portion may be comprised of the same material as the outer tubular portion.

The inner tubular portion may have an outer diameter of between 10 and 30 mm, preferably between, 12 and 20 mm, more preferably between 14 and 18 mm, for example between 15 and 16 mm, such as 15.5 mm.

The inner tubular portion may have an inner diameter of between 6 and 26 mm, preferably between 8 and 20 mm, more preferably between 10 and 16 mm, for example between 12 and 13 mm, such as 12.3 mm.

The inner tubular portion may have a wall thickness (as measured from the radially outer wall to the radially inner wall of between 1 and 2 mm, preferably between 1.2 and 1.8 mm, more preferably between 1.5 and 1.7 mm, for example between 1.6 mm.

The inner tubular portion may have a length of between 30 and 100 mm, preferably between 30 and 50 mm, more preferably between 40 and 45 mm, for example between 42 and 43 mm, such as 42.5 mm.

The difference between the inner diameter of the inner tubular portion and the aperture in the end wall (i.e. twice the radial extent of the end wall) may be between 2 and 10 mm, preferably between 3 and 8 mm, more preferably between 4 and 6 mm, for example between 4 and 5 mm such as 4.4 mm.

The intermediate tubular portion may be comprised of plastic. The intermediate tubular portion may be comprised of a plastic with a Shore hardness as measured using a Shore Durometer “A” of between 40 and 95 (i.e. between 40A and 95A). The intermediate tubular portion may be comprised of a plastic with a Shore hardness as measured using a Shore Durometer “A” of between 40 and 50 (i.e. between 40A and 50A). The intermediate tubular portion may be comprised of a plastic with a Shore hardness as measured using a Shore Durometer “A” of between 50 and 60 (i.e. between 50A and 60A). The intermediate tubular portion may be comprised of a plastic with a Shore hardness as measured using a Shore Durometer “A” of between 60 and 70 (i.e. between 60A and 70A). The intermediate tubular portion may be comprised of a plastic with a Shore hardness as measured using a Shore Durometer “A” of between 70 and 80 (i.e. between 70A and 80A). The intermediate tubular portion may be comprised of a plastic with a Shore hardness as measured using a Shore Durometer “A” of between 80 and 95 (i.e. between 80A and 95A). The intermediate tubular portion may be comprised of a plastic with a Shore hardness as measured using a Shore Durometer “A” of between 50 and 80 (i.e. between 50A and 80A). The intermediate tubular portion may be comprised of a plastic with a Shore hardness as measured using a Shore Durometer “A” of no more than 50 (i.e. 50A). The intermediate tubular portion may be comprised of a plastic with a Shore hardness as measured using a Shore Durometer “A” of no more than 60 (i.e. 60A). The intermediate tubular portion may be comprised of a plastic with a Shore hardness as measured using a Shore Durometer “A” of no more than 70 (i.e. 70A). The intermediate tubular portion may be comprised of a plastic with a Shore hardness as measured using a Shore Durometer “A” of no more than 80 (i.e. 80A). The intermediate tubular portion may be comprised of silicone.

In a preferred embodiment, there is provided a catheter assembly comprising radially nested inner, intermediate and outer tubular portions, wherein the intermediate tubular portion is arranged between and adjacent to the inner and outer tubular portions, the inner tubular portion comprising a first material, the intermediate tubular portion comprising a second material and the outer tubular portion comprising a third material, wherein the second material is softer than the first and third materials, wherein the intermediate tubular portion has a Shore hardness of between 40 and 95 as measured using a Shore Durometer “A”.

A distal end of the intermediate tubular portion may extend to the same axial extent as the inner tubular portion. A proximate end of the intermediate tubular portion may extend axially beyond the proximal end of the inner tubular portion. The section of the intermediate tubular portion extending axially beyond the proximal end of the inner tubular portion may define a wetting agent chamber. The wetting agent chamber may be further defined by a proximal end wall of the inner tubular portion.

In a preferred embodiment, there is provided a catheter assembly comprising radially nested inner, intermediate and outer tubular portions, wherein the intermediate tubular portion is arranged between and adjacent to the inner and outer tubular portions, the inner tubular portion comprising a first material, the intermediate tubular portion comprising a second material and the outer tubular portion comprising a third material, wherein the second material is softer than the first and third materials, wherein a proximal end of the intermediate tubular portion extends axially beyond a proximal end of the inner tubular portion, the section extending beyond the inner tubular portion defining a wetting agent chamber.

The wetting agent chamber may have a volume of between 500 and 800 mm³, preferably between 600 and 700 mm³, more preferably between 630 and 700 mm³, for example between 650 and 680 mm³. The wetting agent may be a gel, for example a silicone gel.

The section of the intermediate tubular portion extending axially beyond the proximal end of the inner tubular portion may taper such that the diameter decreases towards the proximal end thereof. The proximal end of the intermediate tubular portion, e.g. a proximal end wall of the intermediate tubular portion may comprise an aperture. The aperture may be configured to allow a/the catheter tube to extend therethrough. The aperture may be shaped and/or sized to provide an engineering fit to a/the catheter tube. The aperture may provide an interference fit to the catheter tube.

By utilising the intermediate tubular portion for the wetting agent storage chamber the number of components is further reduced and the inherent softness of the intermediate material can be utilised to provide a seal against the catheter tube.

The intermediate tubular portion may have a wall thickness (as measured from the radially outer wall to the radially inner wall) of between 0.5 and 1 mm, preferably between 0.7 and 0.9 mm, more preferably between 0.75 and 0.75 mm, for example between 0.8 mm.

The intermediate tubular portion may have a length of between 30 and 150 mm, preferably between 40 and 90 mm, more preferably between 55 and 75 mm, for example between 60 and 70 mm, such as 65 mm.

The radially inner wall of the intermediate tubular portion may have a corresponding profile to the radially outer wall of the inner tubular portion. The radially inner wall of the intermediate tubular portion may be sized and/or shaped so as to provide an engineering fit, such as an interference fit, between the inner and intermediate tubular portions.

The radially inner wall of the intermediate tubular portion may have a diameter at least 0.01 mm, at least 0.05 mm, at least 0.1 mm less than the diameter of the radially outer wall of inner tubular portion.

The radially outer wall of the intermediate tubular portion may have a diameter at least 0.01 mm, at least 0.05 mm, at least 0.1 mm greater than the diameter of the radially inner wall of outer tubular portion.

The catheter assembly may comprise a cap. The cap may define a portion of the external housing. The cap may be detachable. The cap may be attached to the remainder of the external housing. The cap may be hingibly mounted to the outer tubular portion. The hingibly mounted at a distal end of the outer tubular portion. The hinge may be a moulded strap hinge. The hinge may be an integral coined hinge. The outer tubular portion and the cap may be integrally formed, that is moulded as a single component, i.e. a single injection moulding.

The cap may comprise an annular projection. The annular projection may be provided on an inner surface of the cap. The annular projection may be an annular ring. The annular projection/ring may have an outer diameter less than or equal to the outer diameter of the intermediate tubular portion. The annular projection/ring may have an inner diameter greater than or equal to the inner diameter of the intermediate tubular portion. Preferably the outer diameter of the annular projection/ring is less than the outer diameter of the intermediate tubular portion and the inner diameter of the annular projection/ring is greater than the inner diameter of the intermediate tubular portion. When the cap is in a closed configuration, the annular projection may be configured to bear against the intermediate tubular portion. When the cap is in a closed configuration, the annular projection may be configured to bear axially against the intermediate tubular portion. The annular projection may bear against a distal end of the intermediate tubular portion.

Advantageously, the aforementioned arrangement of the cap and intermediate tubular portion allows the latter to also function as a seal for the catheter assembly, reducing the number of components required. It also helps to ensure that the catheter is resealable—albeit in a non-sterile manner (after an initial sterile seal is broken)—which may be beneficial if the user wishes to temporarily postpone use of the catheter, or to secure the catheter in a leak resistant manner for later disposal after use.

Accordingly, in a preferred embodiment there is provided a catheter assembly comprising radially nested inner, intermediate and outer tubular portions, wherein the intermediate tubular portion is arranged between and adjacent to the inner and outer tubular portions, the inner tubular portion comprising a first material, the intermediate tubular portion comprising a second material and the outer tubular portion comprising a third material, wherein the second material is softer than the first and third materials, wherein the outer tubular portion is an external housing and the external housing further comprises a cap, wherein the cap comprises an annular ring configured to bear against the intermediate tubular portion at a distal end thereof.

According to a further broad aspect of the invention there is provided a method of manufacturing a catheter assembly. The catheter assembly may comprise an inner tubular portion. The catheter assembly may comprise an intermediate tubular portion. The catheter assembly may comprise an outer tubular portion. The inner tubular portion may comprise a first material. The intermediate tubular portion may comprise a second material. The outer tubular portion may comprise a third material. The second material may be softer than the first material. The second material may be softer than the third material. The method may comprise inserting the intermediate tubular portion into the outer tubular portion. The method may comprise inserting the inner tubular portion into the intermediate tubular portion.

Accordingly, in a second aspect of the invention the is provided a method of manufacturing a catheter assembly comprising inner, intermediate and outer tubular portions, the inner tubular portion comprising a first material, the intermediate tubular portion comprising a second material and the outer tubular portion comprising a third material, wherein the second material is softer than the first and third materials, the method comprising: inserting the intermediate tubular portion into the outer tubular portion, and inserting the inner tubular portion into the intermediate tubular portion.

Advantageously, the method set out above provides for an improved manufacture of a catheter assembly, reducing the force required to assemble to parts and reducing the risk of damage to a component.

A catheter assembly of the second aspect may be the catheter assembly of the first aspect, optionally including any optional features thereof.

The method may comprise: inserting the intermediate tubular portion into the outer tubular portion, before inserting the inner tubular portion into the intermediate tubular portion.

The step of inserting the inner tubular portion into the intermediate tubular portion after inserting the intermediate tubular portion into the outer tubular portion may lock the intermediate tubular portion into the outer tubular portion. For example, inserting the inner tubular member may bring the support of the inner tubular portion into alignment with one or more of the projections and depressions in the intermediate tubular portion and the outer tubular portion, locking one or more projections into one or more respective depressions.

The method may further comprise the step of inserting a catheter into the inner tubular portion.

The method may comprise the step of closing a cap to seal a catheter within the catheter assembly. The cap may comprise an annular ring, the annular ring may be brought to bear against the intermediate tubular portion. Bringing the annular ring to bear against the intermediate tubular portion may cause radial expansion of the intermediate tubular portion against the inner and outer tubular portions. Expansion of the intermediate tubular portion may seal the catheter assembly. The catheter assembly may be sterilised. The seal may maintain a sterile seal.

### DETAILED DESCRIPTION OF THE INVENTION

In order that the invention may be more clearly understood one or more embodiments thereof will now be described, by way of example only, with reference to the accompanying drawings, of which:

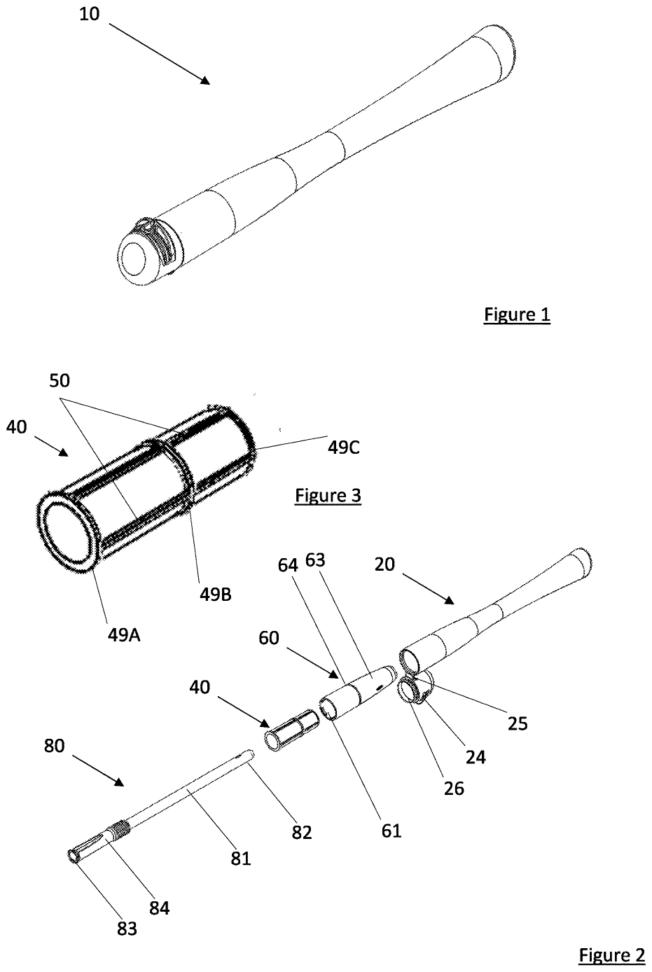

FIG. 1 shows a perspective view of a catheter assembly according to an embodiment of the disclosure in a closed or sealed configuration;

FIG. 2 shows an exploded view of the catheter assembly of FIG. 1 in a closed or sealed configuration;

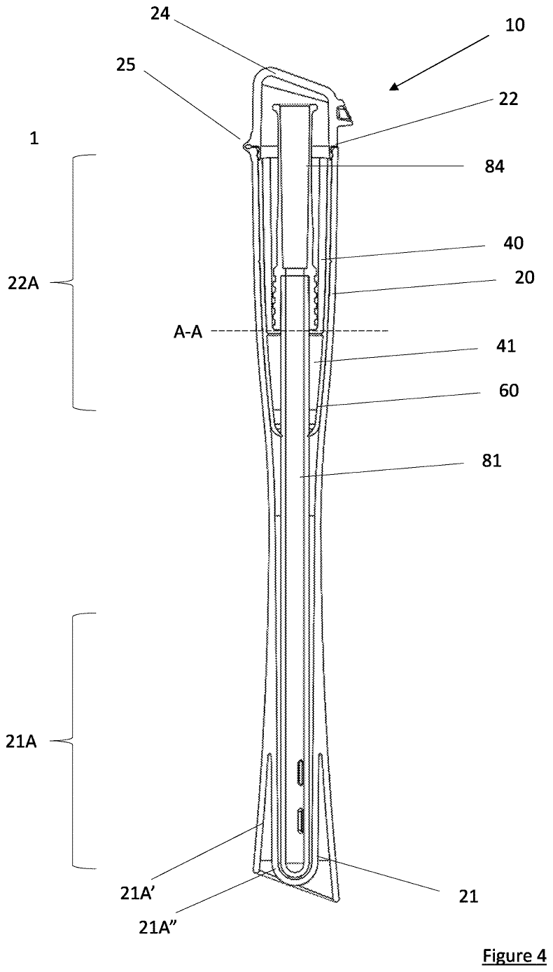

FIG. 3 shows a perspective view of the inner tubular portion of the catheter assembly of FIG. 1;

FIG. 4 shows a longitudinal section of the catheter assembly of FIG. 1 in a closed configuration;

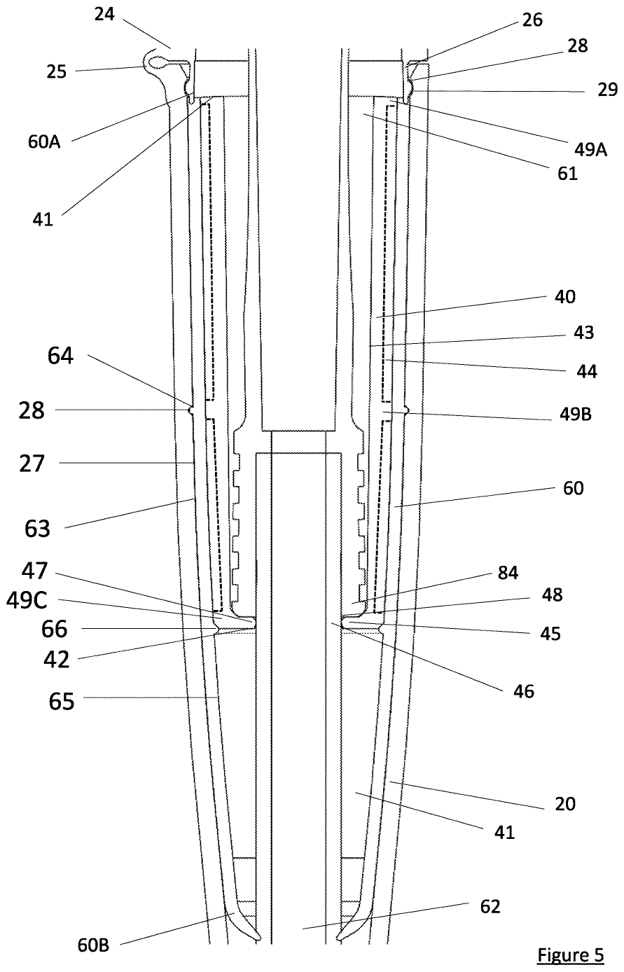

FIG. 5 shows an enlarged longitudinal sectional view of the distal end of the catheter assembly of FIG. 3 detailing a catheter holder and wetting agent chamber;

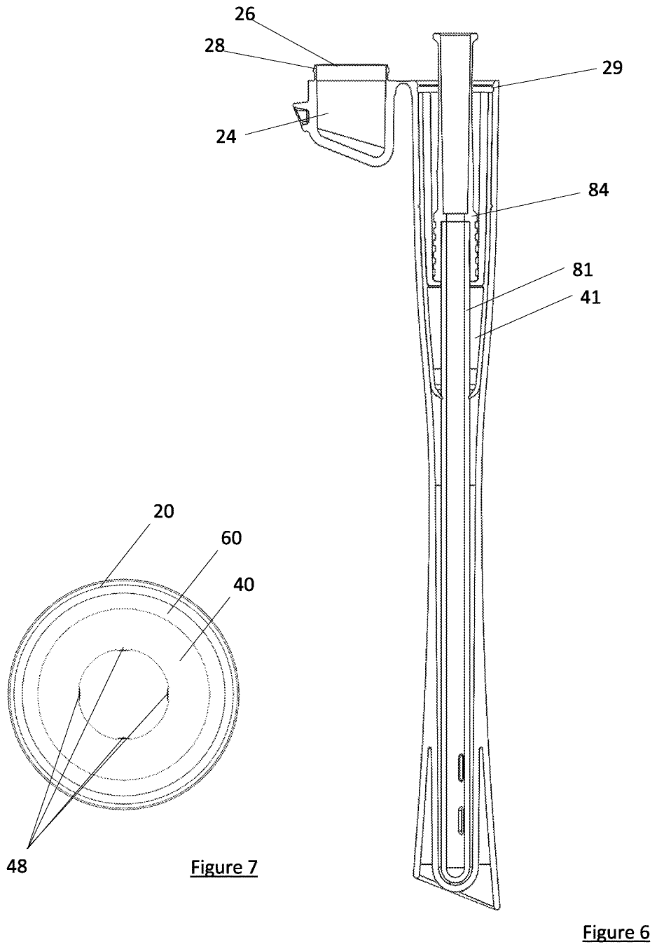

FIG. 6 shows a longitudinal sectional view of the catheter assembly of FIG. 1 in an open configuration; and

FIG. 7 shows a transverse sectional view of the catheter assembly of FIG. 1 along line A-A as shown in FIG. 4.

With reference to the figures (in particular FIGS. 1 to 3), the catheter assembly 10 comprises a catheter 80 and an external housing or case which in this embodiment is in the form of an outer tubular portion 20, an inner tubular portion 40 and an intermediate tubular portion 60. The outer tubular portion 20, inner tubular portion 40, intermediate tubular portion 60 and catheter 80 are concentrically arranged such that the catheter 80 is located within the inner tubular portion 40 which is located within the intermediate tubular portion 60, which in turn is located within the outer tubular portion 20 in a radially nested configuration.

The catheter assembly 10 may be configured such that the catheter 80 may be wetted or lubricated prior to being withdrawn from the external housing 20. A wetting agent is used to wet the catheter 80 prior to use and may be held in a wetting agent storage chamber 41 which in this embodiment is defined by the inner 40 and intermediate 60 tubular portions. The wetting agent may be delivered to the catheter 80 via the wetting agent storage chamber. The wetting agent may be water (in particular where the catheter has a hydrophilic surface) or some other suitable agent, as well known in the art. Preferably, the wetting agent is a gel, e.g. a silicone gel or similar viscous lubricant, which can be used with a catheter that does not include a hydrophilic surface.

Throughout the present application the catheter 80 is used to define an orientation of the catheter assembly 10. The catheter 80 comprises a catheter tube 81 terminating in an insertion end 82 for insertion into the urethra and an opposing drainage end 83. The drainage end 83 comprises a funnel 84 which may comprise a textured outer surface to improve grip. The insertion end 82 of the catheter defines a proximal end of the catheter assembly and the drainage end 83 defines a distal end of the catheter assembly 10.

Referring to FIGS. 2 to 5 the outer tubular portion 20 defines the external housing 20 having an elongate cylindrical shape with a closed proximal end 21 and an open distal end 22. The external housing 20 is slightly waisted, narrowing in diameter at a midpoint relative to the two ends 21,22 where it flares. The distal flared section 22A accommodates a wetting agent storage chamber 41 as will be described in greater detail below. The proximal flared section 21A is formed by a divergence of the outer tubular portion 20, an inner portion 21A′ extending with a constant diameter to a hemispherical end 23 and an outer portion 21A″ which flares out.

A cap 24 is provided at the distal end 22 of the external housing 20, the cap 24 of this embodiment is attached to the remainder of the external housing 20 by an integral molded strap hinge 25, but those skilled in the art will readily envisage alternative means of connection for an openable cap 24. An inner face of the cap 24 (that is the surface of the cap which faces the remainder of the catheter assembly when the cap is in the closed configuration) is provided with a projection in the shape of an annular ring 26. The annular ring 26 extends axially from the inner face of the cap 24 towards the remainder of the catheter assembly 10 and has a diameter approximately equal to that of the intermediate tubular portion 60, the interaction between the annular ring 26 and intermediate tubular portion 60 provides a seal for the catheter assembly 10 as will be elaborated upon below.

The radially inner wall 27 of the outer tubular portion 20 is provided with a groove 28 which extends circumferentially around the inner wall. The groove 28 is positioned such that when the intermediate tubular portion 60 is fully inserted into the outer tubular portion 20, the groove 28 receives a rib on the intermediate tubular portion 60 as will be elaborated upon below.

The outer tubular portion 20 is comprised of a plastics material which is relatively harder than the material from which the intermediate tubular portion 60 is comprised. That is the Shore hardness of the material from which the outer tubular portion is comprised has a higher Shore hardness number as measured under the conditions set forth in ISO868. In the present embodiment, the outer tubular portion 20 is comprised of comprised of polyvinylchloride with a Shore hardness as measured using a Shore Durometer “A” of between 80 and 95, or as measured using a Shore Durometer “D” of between 40 and 90. Obviously in light of this disclosure, those skilled in the art will be readily able to choose alternative materials for this and other portions of the catheter assembly.

As best seen in FIG. 2, the intermediate tubular portion 60 has an overall bullet-shape, gradually tapering such that the diameter decreases towards the proximal end. The intermediate tubular portion 60 is open at both ends, at the distal end 60A is an opening 61 is shaped and sized to receive the inner tubular portion 40 as will the elaborated upon below. At the proximal end 60B there is an aperture 62 sized and shaped so as to provide an engineering fit to the catheter tube 81 as will be elaborated upon below.

Located on a radially outer wall 63 of the intermediate tubular portion 60 is a rib 64, the rib extends circumferentially around the intermediate tubular portion and projects radially outward.

The opposing radially inner wall 65 of the intermediate tubular portion 60 there is provided an insertion stop 66 formed by a circumferentially extending rib, the function of the insertion stop will be elaborated upon below.

The intermediate tubular portion 60 is comprised of a plastics material which is relatively softer than the material from which the outer 20 and inner 40 tubular portions are comprised. That is the Shore hardness of the material from which this intermediate tubular portion is comprise has a lower Shore hardness number as measured under the conditions set forth in ISO868. In the present embodiment, the intermediate tubular portion 40 in comprised of silicone with a Shore hardness of between 40 and 95 as measured using a Shore Durometer “A”.

A best seen in FIGS. 3 and 5, the inner tubular portion 40 has a tubular form with two open ends 41,42 and radially inner 43 and outer 44 walls, the distal end 41 of the inner tubular portion is shaped and sized to receive the catheter funnel 84. The proximal end 42 of the inner tubular portion comprises an end wall 45 with a circular aperture 46 concentrically arranged thereon. The aperture 46 has an inner wall 47 which extends from an interior to an exterior of the inner tubular portion, the inner wall is provided with a plurality of pips 48 (as best seen in FIG. 7) having a hemispherical shape which are configured to bear against and hold the catheter tube 81 In this embodiment there are four pips, but there may be fewer or more—preferably there are at least three pips, to centrally align the catheter tube.

A best seen in FIG. 3, the radially outer wall 44 of the inner tubular portion is provided with a plurality of ribs projecting radially outward, three ribs 49 extend circumferentially around the inner tubular portion, a first circumferential rib 49A is provided at the distal end 41 of the inner tubular portion, a second circumferential rib 49B is provided approximately two thirds of the way from the distal end 41 to the proximal end 42, and a third circumferential rib 49B is provided at the proximal end 42. Extending axially between the first 49A and third 49C circumferential ribs are four axial ribs 50 arranged equidistantly around the circumference of the inner tubular portion 40. As will be expanded upon below the second circumferential rib 49B functions as a support for corresponding projections and recesses on the outer and intermediate tubular portions. The four axial ribs 50 reinforce the inner tubular portion and provide additional support to the intermediate tubular portion.

The inner tubular portion 40 is comprised of a plastics material which is relatively harder than the material from which the intermediate tubular portion 60 is comprised. That is the Shore hardness of the material from which the inner tubular portion is comprised has a higher Shore hardness number as measured under the conditions set forth in ISO868. In the present embodiment, the inner tubular portion 20 is comprised of comprised of polyvinylchloride with a Shore hardness as measured using a Shore Durometer “A” of between 80 and 95, or as measured using a Shore Durometer “D” of between 40 and 90.

To assemble the components into the catheter assembly 10 the intermediate tubular portion 60 is first inserted proximal end first into the open distal end 22 of the outer tubular portion 20 until the rib 64 extending from the outer wall of the intermediate tubular portion 60 is received by the groove 28 in the inner wall of the outer tubular portion 20. The groove 28 and rib 64 are positioned such that when the rib 64 is arranged in the groove 28, the distal end of the intermediate tubular portion 20 is slightly recessed into (i.e. set back from the end of) the outer tubular portion 20. As the intermediate tubular portion 60 is comprised of silicone, it is relatively easily elastically deformed upon insertion making the insertion easier and reducing the risk of damage to the components as compared to when harder materials are used, and thus greater force is required to temporarily deform the components on assembly.

Next the inner tubular portion 40 is inserted proximal end 42 first into the open distal end of the intermediate tubular portion 60 until the proximal end 42 abuts the insertion stop 66. The insertion stop 66 is positioned such that when the proximal end 42 of the inner tubular portion 40 abuts the insertion stop 66 the distal end 41 is axially aligned with the distal end of the intermediate tubular portion.

The inner tubular portion 40 is sized such that the outer diameter is slightly larger than the inner diameter of the intermediate tubular portion 60, in the present embodiment the diameter is at least 0.01 mm greater. This causes the intermediate tubular portion 60 to be slightly deformed thereby helping to retain the inner tubular portion 40 within and also to retain the intermediate tubular portion 60 within the outer tubular portion 20. The provision of ribs 49, 50 on the radially outer wall 44 of the inner tubular portion reduce the surface area in contact with the intermediate tubular portion 60 thereby reducing friction between the two parts upon insertion.

Once fully inserted the second circumferential rib (or “support”) 49B is axially aligned with the rib 64 and groove 28 in the intermediate and outer tubular portions respectively, the second circumferential rib 49B bears against the wall opposing the rib 64 thereby preventing it from becoming dislodged and thereby locking the intermediate tubular portion 60 into position with respect to the outer tubular portion 20.

The three tubular portions 20, 40, 60 are thus assembled and form a case for the catheter 80. The provision of an intermediate tubular portion 60 made of a softer material sandwiched between two harder tubular portions makes for an improved manufacturing process. The softer intermediate tubular portion is more readily deformed, thereby reducing the amount of force required to assemble the components whilst the outer 20 and inner 40 tubular portions provide strength to the case.

As assembled the proximal end of the intermediate tubular portion 60 extends axially beyond the proximal end 42 of the inner tubular member 40 and thus the end wall 45 and radially inner wall 65 define a wetting agent storage chamber 41. The catheter 80 is inserted insertion end 82 first into the case such that the catheter tube 81 passes through the circular aperture 46 in the inner tubular portion 40 and the aperture 62 in the proximal end of the intermediate tubular portion 60 until the funnel 84 abuts the end wall 45.

The diameter to the aperture 62 is less than the diameter of the catheter tube 81 which prevents or at least restricts leakage of the wetting agent, in this embodiment a lubricating silicone gel, onto the remainder of the catheter tube beyond the wetting agent storage chamber 41. The pips 48 ensure that the catheter is held centrally within the case. Whilst the pips 48 prevent the wetting agent storage chamber being fully sealed, the small separation between the catheter tube 81 and inner wall 47 of the aperture 46 caused by the pips 48 (typically of the order of 0.07 mm) and the viscosity of the lubricating gel, the gel does not, or is significantly restricted in leaking from the wetting agent storage chamber via the aperture 46.

To ensure the sterility of the catheter prior to use the catheter assembly is then sealed using the integrally provided cap 24. The cap 24 is pivoted about the hinge 25 such that the annular ring 26 is inserted into the open distal end 22 of the outer tubular portion 20. A radially outer wall 27 of the annular ring 26 is provided with a bulge 28 which is spaced to engage with a corresponding recess 29 on an inner edge of the open distal end 22 thereby securing the cap 24 in place. Distal from the remainder of the cap 24 is a terminal end 30 of the annular ring 26, the terminal end 30 bears against and deforms the distal end 60A of the intermediate tubular portion 60. This causes the distal end 60A do deform radially, and bear against the outer 20 and inner 40 tubular portions thereby sealing the catheter assembly 10.

The wetting agent may be introduced during assembly, or subsequently, via a port (not shown) in the outer tubular member 20, allowing access to the wetting agent chamber. Once sealed with the wetting agent in its chamber, and the port closed, the catheter can be sterilised by known means (or possibly each part of the assembly can be sterilised prior to assembly and the assembly can take place in a sterile environment), thus, once sealed, the seal maintains a sterile environment within the case of the catheter assembly.

To use the catheter 80, the user, or, a health care professional, or caregiver, opens the cap 24 on the catheter assembly 10 as shown in FIG. 6. In some embodiments the cap may comprise a frangible indicator or the like to show that the sterile seal has been broken. The user may then withdraw the catheter 80 by pulling on the funnel 84. Upon withdrawal of the catheter 80 the catheter tube 81 passes through the wetting agent storage chamber 41 whereupon the catheter tube 81 is coated with the lubricating gel. The pips 48 ensure a consistent even coating of lubricating gel is provided along the length of the catheter tube 81. This is particularly beneficial as the insertion end 82 of the catheter, which is inserted into a canal, vessel, passageway, body cavity, etc. for removal of fluid therefrom first and thus most needing of lubrication, is the last part to pass through the wetting agent storage chamber 41 and thus controlled consistent lubrication reduces the risk that the insertion end 82 is unlubricated.

Once the catheter 80 has been used to drain fluid, it can be returned to the catheter assembly 10 for later disposal. The cap 24 can be closed thereby resealing the case and avoid or reduce unwanted leakage of fluid prior to disposal.

The one or more embodiments are described above by way of example only. Many variations are possible without departing from the scope of protection afforded by the appended claims.

For example, whilst the embodiments are all female intermittent urinary catheters, with an exemplary length of between 90 mm to 200 mm. e.g. between 130 mm and 155 mm, such as about 135 mm and the catheter assemblies have a length corresponding to the length of the catheter, such as a closed length of the casing of between 2 mm and 10 mm longer than the length of catheter (e.g. 10-25 cm; between 140 mm and 165 mm, such as 142 mm), it is considered that teachings could be applied to male urinary intermittent catheters (which are typically longer) or even other types of catheter. Similarly, although the embodiments use a lubricating gel, such as silicone gel, the catheter could alternatively have functionalised hydrophilic surfaces which become slippery when wetted with a wetting agent such as water.

1. A catheter assembly comprising radially nested inner, intermediate and outer tubular portions, wherein the intermediate tubular portion is arranged between and adjacent to the inner and outer tubular portions, the inner tubular portion comprising a first material, the intermediate tubular portion comprising a second material and the outer tubular portion comprising a third material, wherein the second material is softer than the first and third materials.

2. A catheter assembly according to claim 1 wherein a case comprises the inner, intermediate and outer tubular portions and the case is configured to receive a catheter.

3. A catheter assembly according to claim 1 or 2 wherein the intermediate tubular portion has a Shore hardness of between 40 and 95 as measured using a Shore Durometer “A”.

4. A catheter assembly according to any preceding claim wherein the intermediate tubular portion comprises silicone.

5. A catheter assembly according to any preceding claim wherein the inner and/or outer tubular portions have a Shore hardness as measured using a Shore Durometer “A” of between 80 and 95, and/or as measured using a Shore Durometer “D” of between 40 and 90.

6. A catheter assembly according to any preceding claim wherein the inner and/or outer tubular portions comprise polyvinylchloride or polypropylene.

7. A catheter assembly according to any preceding claim wherein the outer tubular portion is an external housing.

8. A catheter assembly according to claim 7 wherein the external housing defines a sterile cavity.

9. A catheter assembly according to claim 7 or 8 wherein the external housing further comprises a cap at a distal end thereof.

10. A catheter assembly according to claim 9 wherein the cap comprises an annular ring configured to bear against the intermediate tubular portion at a distal end thereof.

11. A catheter assembly according to any preceding claim wherein one of the outer tubular portion and intermediate tubular portion comprises one or more depressions and the other of the outer tubular portion and intermediate tubular portion comprises one or more projections, the projections arranged in the depressions.

12. A catheter assembly according to claim 11 wherein the inner tubular portion comprises at least one support aligned with the depression and projection.

13. A catheter assembly according to any preceding claim wherein the inner tubular portion comprises a catheter holder.

14. A catheter assembly according to any preceding claim, wherein the inner tubular portion comprises an end wall, the end wall extending radially inward from a proximal end of the inner tubular portion, the end wall being provided with an aperture extending therethrough.

15. A catheter assembly according to claim 14 wherein the aperture is provided with one or more projections configured to bear against a catheter tube.

16. A catheter assembly according to any preceding claim wherein a proximal end of the intermediate tubular portion extends axially beyond a proximal end of the inner tubular portion, the section extending beyond the inner tubular portion defining a wetting agent chamber.

17. A catheter assembly according to claim 16 wherein the proximal end of the intermediate tubular portion comprises an aperture, wherein the aperture is shaped and/or sized to provide an engineering fit to a catheter tube.

18. A method of manufacturing a catheter assembly comprising inner, intermediate and outer tubular portions, the inner tubular portion comprising a first material, the intermediate tubular portion comprising a second material and the outer tubular portion comprising a third material, wherein the second material is softer than the first and third materials, the method comprising:
inserting the intermediate tubular portion into the outer tubular portion,
inserting the inner tubular portion into the intermediate tubular portion.

19. A method of manufacturing a catheter assembly according to claim 18 further comprising the step of inserting a catheter into the inner tubular portion.

20. A method of manufacturing a catheter assembly according to claim 18 or 19 wherein the catheter assembly is a catheter assembly according to any of claims 1 to 17.
